(19) United States
(12) Patent Application Publication
(10) Pub. No.: US 2003/0083697 A1
(43) Pub. Date: May 1, 2003

(54) Implantable neurological lead with low polarization electrode

(76) Inventors: Michael D. Baudino, Coon Rapids, MN (US); Scott J. Brabec, Elk River, MN (US); Paul H. Stypulkowski, North Oaks, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/042,023

(22) Filed: Oct. 25, 2001

(51) Int. Cl. A61N 1/18

(52) U.S. Cl. 607/2

(57) ABSTRACT

An implantable neurological lead is a medical device having at least one low polarization electrode carried on the distal end of the lead. The neurological lead has a proximal end, a distal end, and at least one conductor that is electrically insulated contained in the neurological lead extending from the proximal end to the distal end. The implantable neurological lead is coupleable to an implantable neurological stimulator or implantable neurological monitor. The neurological lead with low polarization electrode has many embodiments and related methods.

### BACKGROUND OF THE INVENTION

This disclosure relates to a medical device and more particularly to implantable neurological electrical stimulators and implantable electrical stimulation leads.

The medical device industry produces a wide variety of electronic and mechanical devices for treating patient medical conditions such as pacemakers, defibrillators, neuro-stimulators and therapeutic substance delivery pumps. Medical devices can be configured to be surgically implanted or connected externally to the patient receiving treatment. Clinicians use medical devices alone or in combination with therapeutic substance therapies and surgery to treat patient medical conditions. For some medical conditions, medical devices provide the best and sometimes the only therapy to restore an individual to a more healthful condition and a fuller life. One type of medical device is an implantable neurological stimulation system that can be used to treat conditions such as pain, movement disorders, pelvic floor disorders, gastroparesis, and a wide variety of other medical conditions. The neurostimulation system typically includes a neurostimulator, a stimulation lead, and an extension such as shown in Medtronic, Inc. brochure “Implantable Neurostimulation System” (1998). More specifically, the neurostimulator system can be an Itrel II® Model 7424 or an Itrel 3® Model 7425 available from Medtronic, Inc. in Minneapolis, Minn. that can be used to treat conditions such as pain, movement disorders and pelvic floor disorders. The neurostimulator is typically connected to a stimulation lead that has one or more electrodes to deliver electrical stimulation to a specific location in the patient's body.

The electrode to tissue interface polarization can influence the electrical stimulation signal delivered to the tissue and the ability to sense physiological activity soon after delivering an electrical stimulation signal. With a constant voltage output device, the current waveform delivered to the tissue is subject to chance depending on the capacitance of the electrode-tissue interface. The electrode tissue interface has been modeled as a series RC circuit where the capacitance portion of the circuit has contributions from both the metal used to inject the charge and the biological tissue. When the electrode tissue interface capacitance is low, the trailing edge can be considerably less resulting in a decreasing amount of current delivered to the tissue and potentially increasing overall power requirements. Since it takes a minimum quantity of charge over a time period to excite the neurological tissue, it could be advantageous to provide uniform charge delivery. In electrodes of the type used for stimulating biological tissue, the polarization after potential, i.e. charge remaining at the electrode interface after a stimulation pulse, is sufficient to mask the low level biological signals that are of interest. In neurological applications, the monitoring of evoked potential is typically done with microelectrodes placed independently and remotely from the stimulation electrode rather than by the same electrode used for stimulation.

Thus, embodiments of the implantable neurological low polarization stimulation system is disclosed to reduce energy requirements and a monitoring system is disclosed to provide for more rapid sensing of post stimulation pulse physiological activity.

### BRIEF SUMMARY OF THE INVENTION

The implantable neurological lead with low polarization electrode has at least one low polarization electrode carried on the distal end of the lead. The neurological lead has a proximal end, a distal end, and at least one conductor that is electrically insulated contained in the neurological lead extending from the proximal end to the distal end. The implantable neurological lead is coupleable to an implantable neurological stimulator or neurological monitor. There are a wide variety of implantable neurological leads with low polarization electrode embodiments and methods relating to the leads.

### BRIEF DESCRIPTION OF THE DRAWINGS

FIG. 1 shows a general environmental view for a neurostimulation system embodiment;

FIG. 2 shows a neurostimulation system embodiment;

FIG. 3a shows a neurostimulation lead embodiment;

FIG. 3b shows another neurostimulation lead embodiment;

FIG. 3c shows a schematic of low polarization electrode materials embodiment;

FIG. 4a (prior art) shows a standard electrode surface embodiment;

FIG. 4b shows a low polarization electrode surface embodiment;

FIG. 4c shows another low polarization electrode with macro surface porosity embodiment;

FIG. 5a (prior art) shows a voltage waveform recorded from a standard stimulation electrode delivered by a constant voltage source;

FIG. 5b shows a voltage waveform recorded from a low polarization stimulation electrode delivered by a constant voltage source embodiment;

FIG. 5c (prior art) shows a current waveform recorded from a standard stimulation electrode delivered by a constant voltage source;

FIG. 5d shows a current waveform recorded from a low polarization stimulation electrode delivered by a constant voltage source embodiment;

FIG. 6 shows a method of delivering a substantially constant current neurostimulation waveform from a constant voltage neurostimulator embodiment;

FIG. 7 shows a method of sensing post neurostimulation waveform physiological activity through a stimulation electrode embodiment; and,

FIG. 8 shows a method for manufacturing a neurological lead with a low polarization electrode embodiment.

### DETAILED DESCRIPTION OF THE PREFERRED EMBODIMENTS

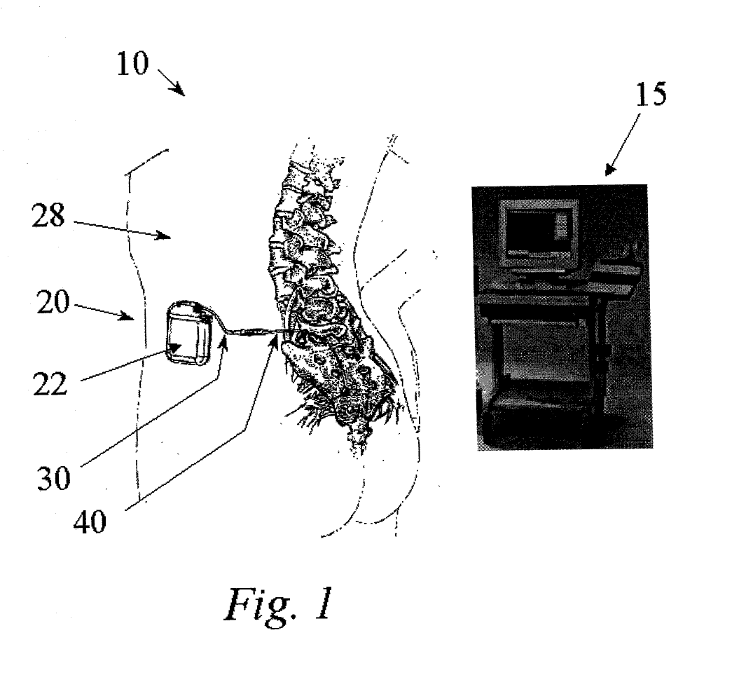

FIG. 1 shows a general environmental view 10 for an implantable neurostimulation system embodiment. Neurostimulation systems are used to treat conditions such as pain, movement disorders, pelvic floor disorders, gastroparesis, and a wide variety of other medical conditions. The neurostimulation system 20 includes a neurostimulator 22 such as an Itrel II® Model 7424 or an Itrel 3® Model 7425 available from Medtronic, Inc. in Minneapolis, Minn., a stimulation lead extension 30, and a stimulation lead 40. The neurostimulator 22 is typically implanted subcutaneously in the patient's body 28 at a location selected by the clinician. The stimulation lead 40 is typically fixed in place near the location selected by the clinician using a device such as the adjustable anchor.

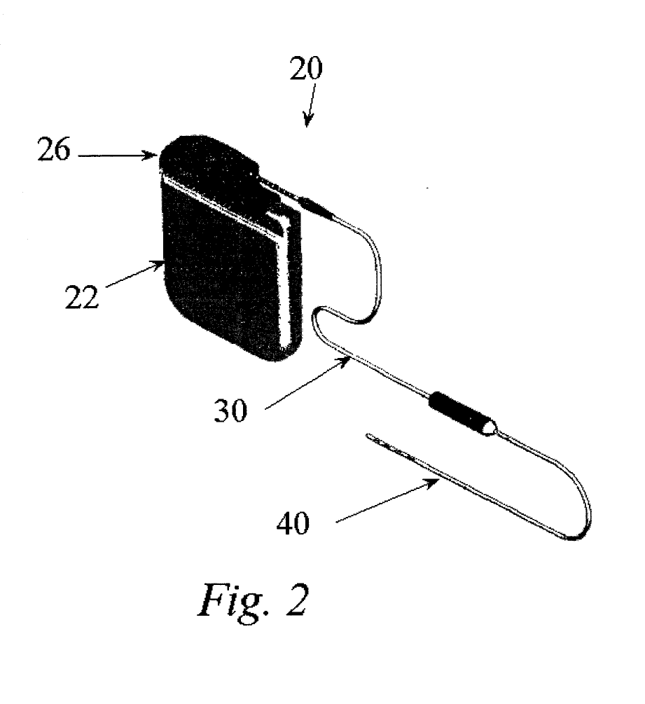

FIG. 2 shows an implantable neurostimulation system 20 comprising an implantable neurostimulator 22, as stimulation lead 40, and a lead extension 30. The implantable neurostimulator 22 has a housing, a power supply carried in the housing 24, and stimulation electronics coupled to the battery and coupled to a connector block 26, which is also known as a terminal block. The stimulation lead 40 has a lead proximal end 45, a lead distal end 41 and a lead body 43. The lead proximal end 45 has at least one electrical connector 46 (also known as electrical terminals) and the lead distal end 41 has at least one stimulation electrode 42. There is at least one lead conductor 44 contained in the lead body 43 that is electrically connecting the electrical connector 46 to the stimulation electrode 42.

An implantable neurological low polarization stimulation or monitoring system comprises an implantable neurological stimulator 22 or neurological monitor, an implantable neurological lead 40, and at least one low polarization electrode 42. The implantable neurological stimulator 22 can be a Medtronic Itrel II® Model 7424 or an Itrel 3® Model 7425 or the like, both of which are commercially available. The neurological monitor 15 can be a Medtronic Neurodiagnostics Keypoint monitoring system.

The implantable neurological lead 40 comprises a lead proximal end 45, a lead distal end 41, at least one conductor 44, at least on low polarizing electrode 42, and at least one electrical connector 46. The lead proximal end 45 contains at least one electrical connector 46 that couples to the implantable neurological stimulator 22 or neurological monitor. The lead distal end 41 contains at least one low polarizing electrode 42. The conductor 44 contained in the lead 40 extending from the lead proximal end 45 to the lead distal end 41, the conductor 44 being electrically insulated by a polymer. The polymer could be, but is not limited to, ethylene tetrafluoroethylene (ETFE), polytetrafluoroethylene (PTFE), silicone rubber or polyurethane. Other materials that act as electrical insulators can be used. The electrical connector 46 is carried on the lead proximal end 45 and electrically connected to the conductor 44. The neurological lead 40 can be configured as a neurological stimulation lead, a neurological sensing lead, and a combination of both as a neurological stimulation and sensing lead.

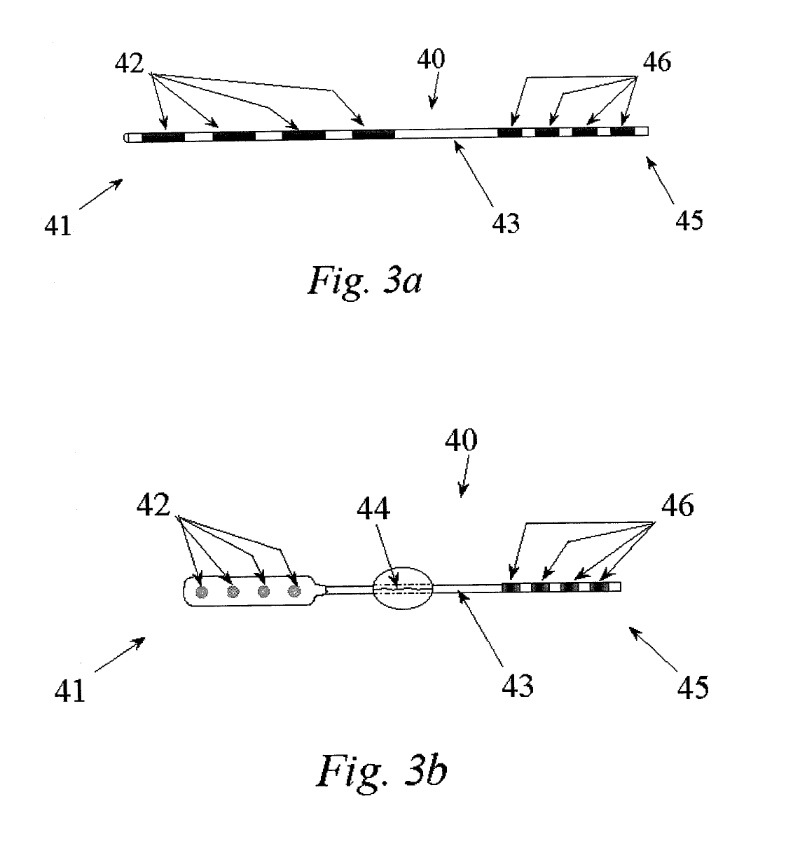

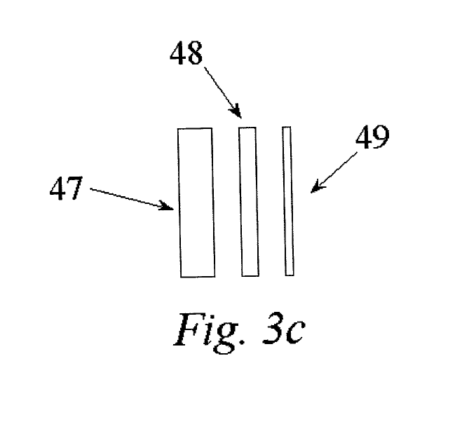

FIGS. 3a and 3b show an implantable neurostimulation lead 40 embodiments that have a lead proximal end 45, a lead distal end 41 and a lead body 43. The lead proximal end 45 has at least one electrical contact 46 for connecting to a lead extension 30 or neurostimulator connector block 26. The lead distal end 41 has at least one stimulation electrode 42, the surface of said stimulation electrode 42 being modified to have low polarization qualities to efficiently transfer electrical charge from the neurostimulator 22 to the nervous tissue of the patient. The lead body 43 carries at least one conductor 44 electrically connecting the lead proximal electrical contact 46 with the lead distal end 41 stimulation electrode 42.

The lead body 43 can be composed of a wide variety of materials and configurations. Materials may include, but not be limited to silicone rubber, polyurethane, fluoropolymers and the like. Configurations could include monolumen and multilumen tubings. The conductor 44 that electrical connects the lead proximal end 45 electrical contact 46 with the lead distal end 41 stimulation electrode 42 can be composed of a wide variety of material and configurations. Materials may include, but not be limited to MP35N, silver drawn filled tubing (Ag-DFT), Platinum iridium alloys, platinum and the like. Configurations could include stranded, braided or solid wire configured in linear or helical coil arrangements.

The at least one low polarization electrode 42 is carried on the lead distal end 41 and electrically connected to the conductor 44 and serves as a means for a means for electrically coupling to tissue with a low polarization effect resulting in delivering a constant current pulse having a current leading edge and a current trailing edge that are substantially the same. In FIG. 3c, the low polarization electrode 42 has a base material 47, a coating material 49, and can include an intermediate layer 48 between the base material 47 and the coating material 49. The base material 47 is a material typically used for electrical stimulation such as platinum, platinum alloys, titanium, titanium alloys, tantalum, tantalum alloys, stainless steel, stainless steel alloys, iridium, iridium alloys, and the like. The coating material 49 covers a selected portion of the low polarization electrode 42 typically in the range from about 60 percent to 100 percent. The coating material 49 is platinum black or a porous carbide, nitride, carbonitride or oxide layer selected from the group consisting of titanium, vanadium, zirconium, niobium, molybdenum, hafnium, tantalum, iridium, platinum, and tungsten.

The intermediate layer 48 is interposed between the base layer 47 and the coating layer 49. The intermediate layer 48 can be textured to mechanically protect the coating. The intermediate layer 48 texturing is made from sintered particles, such as platinum, platinum iridium, titanium, or such, in the range from about 10 microns to about 50 microns. The lead distal end 41 stimulation electrode 42 is composed of a base material 47 such as platinum or an alloy of platinum iridium, other platinum alloys, titanium, titanium alloys, tantalum, tantalum alloys, stainless steel, stainless steel alloys, iridium, or iridium alloys could be used. Treatments that could be used for surface modifications include porous carbide, nitride, or carbonitrides or oxides selected from titanium, vanadium, zirconium, niobium, molybdenum, hafnium, tantalum, iridium, platinum, or tungsten. In the case of the iridium oxide group, the lead electrode 42 surface modification could be applied after the lead 40 has been manufactured resulting in manufacturing efficiencies.

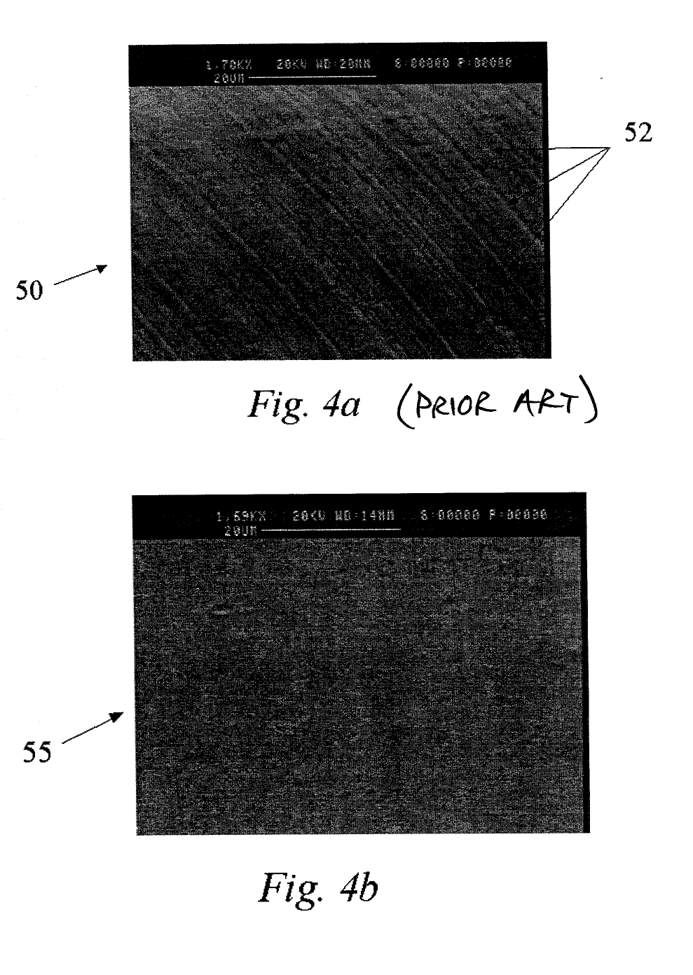

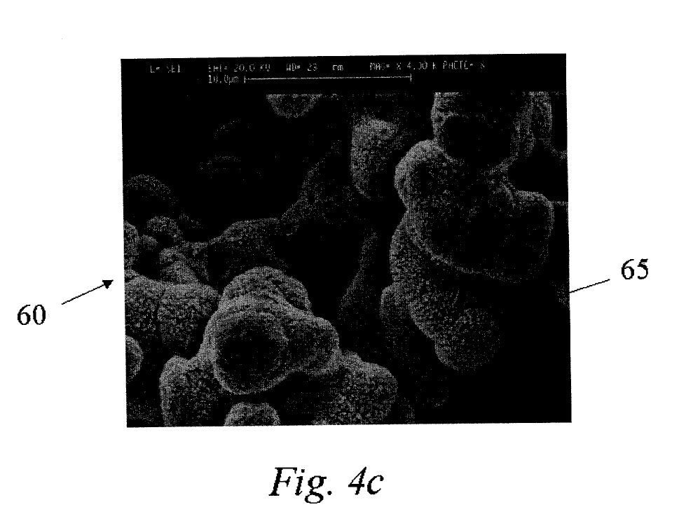

FIG. 4a (prior art) shows a platinum iridium electrode surface 50 at high magnification. Machining marks 52 are evident on the surface of the electrode. FIG. 4b shows a platinum iridium electrode surface 55 that has been modified with an electroplated iridium oxide surface. The machining marks have been covered by the treatment and are no longer evident on the surface. The surface treatment produces a low polarization electrode surface. FIG. 4c shows a platinum iridium electrode surface 60 that has been modified by sintering platinum particles to the surface to create a macroporous surface 65. The surface was modified to include a microporous surface treatment to produce a low polarization electrode 42. The effect of the macro porous region on the electrode serves a two fold purpose. First, it creates additional surface area and second, it provides a protective surface that prevents mechanical removal of the surface treatment due to insertion and manipulation of the lead during introduction into the patient.

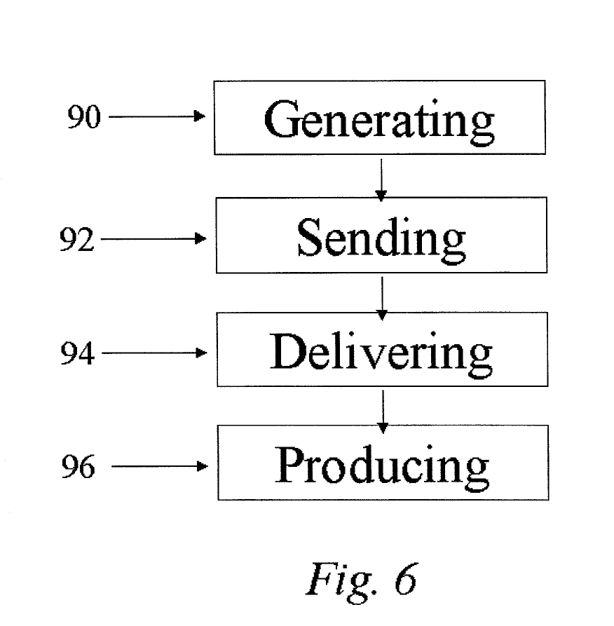

FIG. 6 shows a flow chart for a method of delivering a substantially constant current neurostimulation waveform from a constant voltage neurostimulator. The implantable neurological low polarization stimulation system operates as a method for delivering a substantially constant current neurostimulation waveform from a constant voltage neurostimulator. The method begins with a constant voltage neurological stimulator 22 generating 90 a square stimulation pulse 80 that has substantially constant voltage. The square stimulation pulse 80 having a voltage leading edge 81 and a voltage trailing edge 82. This square stimulation pulse 80 is sent 92 through a neurostimulation lead 40 connected from the constant voltage neurostimulator 22. The square stimulation pulse 80 is delivered 94 through a low polarization electrode 42 coupleable to tissue. The low polarization electrode 42 is connected to the neurostimulation lead 40. A substantially constant current pulse 85 is produced 96 having a current leading edge 86 and a current training edge 87. The current trailing edge 87 is at least 85% of the current leading edge 86 of the substantially constant current pulse 85.

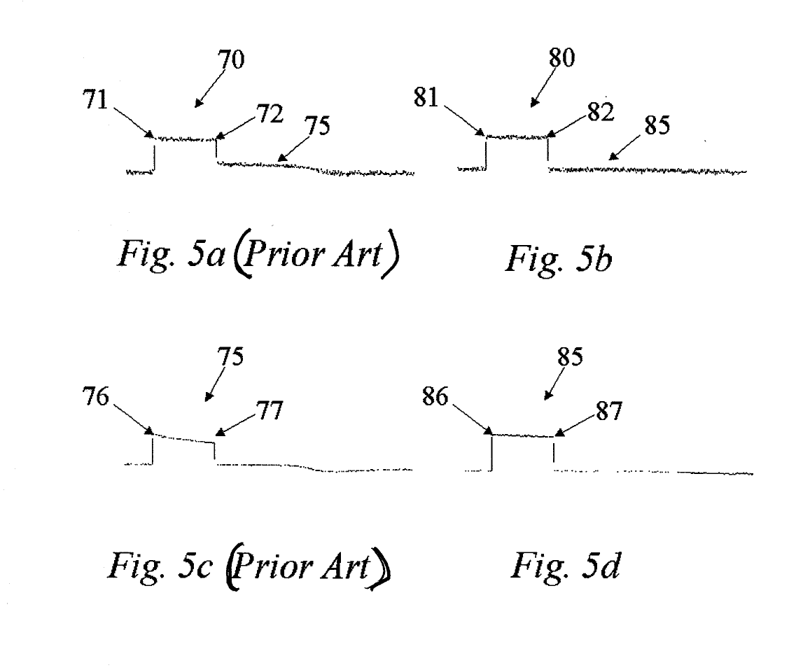

FIG. 5a (prior art) shows a voltage waveform recorded from a standard platinum iridium electrode when it is connected to a constant voltage output neurostimulator 22. The stimulation pulse 70 has a voltage leading edge 71 and a voltage trailing edge 72 separated for a duration of time known as the pulse width where the voltage remains constant. Note the failure of the voltage trailing edge of the pulse to immediately return to the level consistent with the value preceding the voltage leading edge 71. This is known as the post pulse polarization potential 75. FIG. 5b shows a voltage waveform recorded from a low polarization electrodes when it is connected to a constant voltage output stimulator. The stimulation pulse 80 has a voltage leading edge 81 and a voltage trailing edge 82. In this case, the voltage trailing edge 82 of the pulse immediately returns to the waveform preceding the voltage pulse. The post pulse polarization voltage 85 is essentially zero.

FIG. 5c (prior art) shows a current waveform recorded from a standard platinum iridium electrode when it is connected to a constant voltage output neurostimulator 22. The current pulse 75 has a current leading edge 76 and a current trailing edge 77 separated for a duration of time known as the pulse width. With standard electrodes, the current trailing edge 77 value of the pulse is less than the current leading edge 76 due to capacitive influences at the interface. The current waveform is said to droop. The amount of droop depends on the surface area of the electrodes, the stimulation pulse width, and the impedance of the tissue in the immediate vicinity of the electrode. FIG. 5d shows a current waveform recorded from a low polarization electrode 42 when it is connected to a constant voltage output stimulator. The current pulse 85 has a current leading edge 86 and a current trailing edge 87 separated for a duration of time known as the pulse width. The trailing edge 87 in this case is substantially equal to the current leading edge 86 showing constant current flowing through the system.

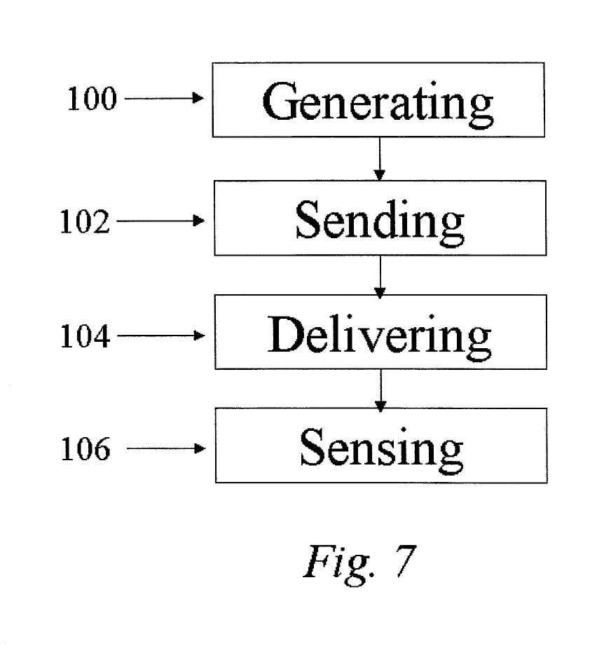

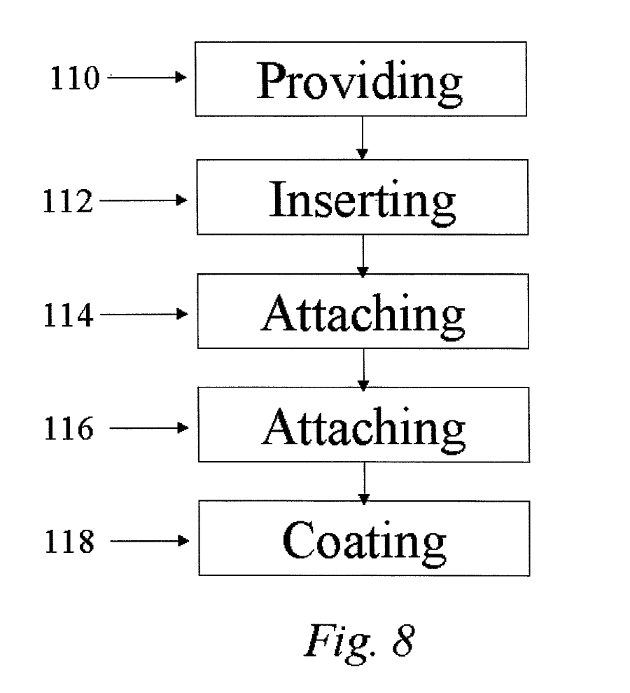

FIG. 7 shows a flow chart for a method of sensing post neurostimulation waveform physiological activity through a stimulation electrode embodiment. In this embodiment, the neurological stimulation system 20 is configured for sensing post neurostimulation waveform physiological activity substantially immediately after delivering a stimulation pulse through the at least one low polarization electrode 42. The method begins by generating 100 a stimulation pulse with a neurostimulator 22. This stimulation pulse is sent 102 through a neurostimulation lead 40 connected to neurostimulator 22. The stimulation pulse is delivered 104 the through an electrode coupleable to tissue, and the electrode is also electrically connected to the neurostimulation lead 40. After delivering the stimulation pulse, post neurostimulation stimulation pulse physiological activity is sensed 106 substantially immediately after delivering the stimulation pulse through a low polarization electrode 42. Sensing 106 post neurostimulation stimulation pulse physiological activity can be done substantially immediately after delivering the stimulation pulse; this can begin within about 20 microseconds after conclusion of the stimulation pulse.

FIG. 8 shows a method for manufacturing a neurological lead with a low polarization electrode. The neurological lead 40 with a low polarization electrode 42 can be manufactured according to the following method. The method begins by providing 110 a lead body 43 having a lead proximal end 45 and a lead distal end 41. At least one conductor 44 is inserted 112 through the lead body 43. At least one terminal 46 is attached 114 to the body proximal end 45 and the at least one terminal 46 is also electrically connected to the at least one conductor 44. The at least one electrode 42 is attached 116 to the lead distal end 41. The at least one electrode 42 has a surface area of at least one square millimeter and also is electrically connected to the at least one conductor 44. The at least one electrode 42 is coated 118 with low polarization coating. The coating for the low polarization can be electroplated iridium oxide or any of the other previously discussed coating applied by an appropriate method.

Thus, embodiments of the implantable neurological low polarization stimulation system is disclosed to reduce energy requirements and a monitoring system is disclosed to provide for more rapid sensing of post stimulation pulse physiological activity. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. An implantable neurological low polarization stimulation or monitoring system, comprising:
an implantable neurological stimulator or monitor;
an implantable neurological lead having a proximal end and a distal end; the implantable neurological stimulation lead being coupled to the implantable neurological stimulator;
at least one conductor contained in the lead extending from the lead proximal end to the distal end, the conductor being electrically insulated;
at least one electrical connector carried on the proximal end and electrically connected to the conductor; and,
at least one low polarization electrode carried on the lead distal end and electrically connected to the conductor.

2. The implantable neurological system as in claim 1 wherein the low polarization electrode has a base material and a coating material.

3. The implantable neurological system as in claim 2 wherein the base material is selected from the group consisting of platinum, platinum alloys, titanium, titanium alloys, tantalum, tantalum alloys, stainless steel, stainless steel alloys, iridium, and iridium alloys.

4. The implantable neurological system as in claim 2 wherein the coating material covers a selected portion of the low polarization electrode.

5. The implantable neurological system as in claim 4 wherein the selected portion is in the range from about 60 percent to about 100 percent.

6. The implantable neurological system as in claim 2 wherein the coating material is platinum black or a porous carbide, nitride, carbonitride or oxide layer selected from the group consisting of titanium, vanadium, zirconium, niobium, molybdenum, hafnium, tantalum, iridium, platinum, and tungsten.

7. The implantable neurological system as in claim 2 further comprising an intermediate layer interposed between the base layer and the coating material.

8. The implantable neurological system as in claim 7 wherein the intermediate layer is textured to mechanically protect the coating material.

9. The implantable neurological system as in claim 8 wherein the intermediate layer texturing is in the range from about 10 microns to about 50 microns.

10. An implantable neurological low polarization stimulation or monitoring system, comprising:
an implantable neurological stimulator or monitor;
an implantable neurological lead having a proximal end and a distal end; the implantable neurological stimulation lead being coupled to the implantable neurological stimulator;
at least one conductor contained in the lead extending from the lead proximal end to the distal end, the conductor being electrically insulated;
at least one electrical connector carried on the proximal end and electrically connected to the conductor; and,
a means for delivering a constant current pulse having a current leading edge and a current trailing edge that are substantially the same, the means for delivering the constant current pulse carried on the lead distal end and electrically connected to the conductor.

11. An implantable neurological stimulation lead with low polarization electrode, comprising:
an implantable neurological lead having a proximal end and a distal end;
at least one conductor contained in the lead extending from the lead proximal end to the distal end, the conductor being electrically insulated;
at least one electrical connector carried on the proximal end and electrically connected to the conductor; and,
at least one low polarization electrode carried on the lead distal end and electrically connected to the conductor.

12. The implantable neurological stimulation lead as in claim 11 wherein the low polarization electrode has a base material and a coating material.

13. The implantable neurological stimulation lead as in claim 12 wherein the base material is selected from the group consisting of platinum, platinum alloys, titanium, titanium alloys, tantalum, tantalum alloys, stainless steel, stainless steel alloys, iridium, and iridium alloys.

14. The implantable neurological stimulation lead as in claim 12 wherein the coating material covers a selected portion of the low polarization electrode.

15. The implantable neurological stimulation lead as in claim 14 wherein the selected portion is in the range from about 60 percent to about 100 percent.

16. The implantable neurological stimulation lead as in claim 12 wherein the coating material is platinum black or a porous carbide, nitride, carbonitride or oxide layer selected from the group consisting of titanium, vanadium, zirconium, niobium, molybdenum, hafnium, tantalum, iridium, platinum, and tungsten.

17. The implantable neurological stimulation lead as in claim 12 further comprising an intermediate layer interposed between the base layer and the coating material.

18. The implantable neurological stimulation lead as in claim 17 wherein the intermediate layer is textured to mechanically protect the coating material.

19. The implantable neurological stimulation lead as in claim 18 wherein the intermediate layer texturing is in the range from about 10 microns to about 50 microns.

20. An implantable neurological stimulation lead with low polarization electrode, comprising:
an implantable neurological lead having a proximal end and a distal end;
at least one conductor contained in the lead extending from the lead proximal end to the distal end, the conductor being electrically insulated;
at least one electrical connector carried on the proximal end and electrically connected to the conductor; and,
at least one low polarization electrode carried on the lead distal end and electrically connected to the conductor,
a means for delivering a constant current pulse having a current leading edge and a current trailing edge that are substantially the same, the means for delivering the constant current pulse carried on the lead distal end and electrically connected to the conductor.

21. An implantable neurological sensing lead with low polarization electrode, comprising:
an implantable neurological lead having a proximal end and a distal end;
at least one conductor contained in the lead extending from the lead proximal end to the distal end, the conductor being electrically insulated;
at least one electrical connector carried on the proximal end and electrically connected to the conductor; and,
at least one low polarization electrode carried on the lead distal end and electrically connected to the conductor.

22. The implantable neurological system as in claim 21 wherein the low polarization electrode has a base material and a coating material.

23. The implantable neurological system as in claim 22 wherein the base material is selected from the group consisting of platinum, platinum alloys, titanium, titanium alloys, tantalum, tantalum alloys, stainless steel, stainless steel alloys, iridium, and iridium alloys.

24. The implantable neurological system as in claim 22 wherein the coating material covers a selected portion of the low polarization electrode.

25. The implantable neurological system as in claim 24 wherein the selected portion is in the range from about 60 percent to about 100 percent.

26. The implantable neurological system as in claim 22 wherein the coating material is platinum black or a porous carbide, nitride, carbonitride or oxide layer selected from the group consisting of titanium, vanadium, zirconium, niobium, molybdenum, hafnium, tantalum, iridium, platinum, and tungsten.

27. The implantable neurological system as in claim 22 further comprising an intermediate layer interposed between the base layer and the coating material.

28. The implantable neurological system as in claim 27 wherein the intermediate layer is textured to mechanically protect the coating material.

29. The implantable neurological system as in claim 28 wherein the intermediate layer texturing is in the range from about 10 microns to about 50 microns.

30. An implantable neurological sensing lead with low polarization electrode, comprising:
an implantable neurological lead having a proximal end and a distal end;
at least one conductor contained in the lead extending from the lead proximal end to the distal end, the conductor being electrically insulated;
at least one electrical connector carried on the proximal end and electrically connected to the conductor; and,
a means for sensing configured to sense a physiological signal within 20 microseconds after delivering a stimulation pulse through the means for sensing without substantial distortion.

31. A low polarization electrode for a neurological lead, comprising:
an implantable neurological lead having a proximal end and a distal end;
at least one conductor contained in the lead extending from the lead proximal end to the distal end, the conductor being electrically insulated;
at least one electrical connector carried on the proximal end and electrically connected to the conductor; and,
at least one low polarization electrode carried on the lead distal end and electrically connected to the conductor.

32. The low polarization electrode as in claim 31 wherein the low polarization electrode has a base material and a coating material.

33. The low polarization electrode as in claim 32 wherein the base material is selected from the group consisting of platinum, platinum alloys, titanium, titanium alloys, tantalum, tantalum alloys, stainless steel, stainless steel alloys, iridium, and iridium alloys.

34. The low polarization electrode as in claim 32 wherein the coating material covers a selected portion of the low polarization electrode.

35. The low polarization electrode as in claim 34 wherein the selected portion is in the range from about 60 percent to about 100 percent.

36. The low polarization electrode as in claim 32 wherein the coating material is platinum black or a porous carbide, nitride, carbonitride or oxide layer selected from the group consisting of titanium, vanadium, zirconium, niobium, molybdenum, hafnium, tantalum, iridium, platinum, and tungsten.

37. The low polarization electrode as in claim 32 further comprising an intermediate layer interposed between the base layer and the coating material.

38. The low polarization electrode as in claim 37 wherein the intermediate layer is textured to mechanically protect the coating material.

39. The low polarization electrode as in claim 38 wherein the intermediate layer texturing is in the range from about 10 microns to about 50 microns.

40. A low polarization electrode for a neurological lead, comprising:
an implantable neurological lead having a proximal end and a distal end;
at least one conductor contained in the lead extending from the lead proximal end to the distal end, the conductor being electrically insulated;
at least one electrical connector carried on the proximal end and electrically connected to the conductor; and,
a means for electrically coupling to tissue with a low polarization effect on the lead distal end.

41. A method of delivering a substantially constant current neurostimulation waveform from a constant voltage neurostimulator, comprising:
generating a square stimulation pulse that has substantially constant voltage with a constant voltage neurostimulator, the square stimulation pulse having a voltage leading edge and a voltage trailing edge;
sending the square stimulation pulse through a neurostimulation lead connected to the constant voltage neurostimulator;
delivering the square stimulation pulse through a low polarization electrode coupleable to tissue, the low polarization electrode being connected to the neurostimulation lead; and,
producing a substantially constant current pulse having a current leading edge and a current training edge,
wherein the current trailing edge is at least 85% of the current leading edge of the substantially constant current pulse.

42. A method of sensing post neurostimulation waveform physiological activity through a stimulation electrode, comprising:
generating a stimulation pulse with a neurostimulator;
sending the stimulation pulse through a neurostimulation lead connected to neurostimulator;
delivering the through an electrode coupleable to tissue, the electrode being connected to the neurostimulation lead;
sensing post neurostimulation stimulation pulse physiological activity substantially immediately after delivering the stimulation pulse through a low polarization electrode.

43. The method as in claim 42 wherein sensing post neurostimulation stimulation pulse physiological activity substantially immediate after delivering the stimulation pulse is done within about 20 microseconds after conclusion of the stimulation pulse.

44. A method for manufacturing a neurological lead with a low polarization electrode, comprising:
providing a lead body having a body proximal end and a body distal end;
inserting at least one conductor through the lead body;
attaching at least one terminal to the body proximal end, the at least one terminal also being electrically connected to the at least one conductor;
attaching at least one electrode to the body distal end, the at least one electrode having a surface area of at least one square millimeter and also being electrically connected to the at least one conductor; and,
coating the at least one electrode with low polarization coating.

45. The method as in claim 44 wherein the low polarization coating is electroplated iridium oxide.
